(19) United States
(12) Patent Application Publication
(10) Pub. No.: US 2005/0009913 A1
(43) Pub. Date: Jan. 13, 2005

(54) Method for inactivating bacteria and leukocytes in thrombocyte suspensions

(76) Inventor: Harald Mohr, Hannover (DE)

Correspondence Address: Hedman & Costigan, 1185 Avenue of the Americas, New York, NY, 10036-2601, US

(21) Appl. No.: 10/499,586

(22) PCT Filed: Dec. 18, 2002
(86) PCT No.: PCT/DE02/04645

(30) Foreign Application Priority Data
Dec. 19, 2001 (DE) 101 62 712.2

(51) Int. Cl. A01N 1/02; A61K 31/195

(52) U.S. Cl. 514/561; 435/2

(57) ABSTRACT

The present invention relates to a method for inactivating bacteria and/or leukocytes in thrombocyte suspensions by adding precursor compounds from which photosensitizers are formed by endogenous synthesis, and subsequent photodynamic treatment.

The subject matter of the present invention is a method for inactivating bacteria and/or leukocytes in thrombocyte suspensions by adding precursor compounds from which photosensitizers are formed by endogenous synthesis, and a photodynamic treatment.

The decontamination of cellular blood products by means of photodynamic methods is known. Photodynamic inactivation of viruses is effected by illuminating the preparation to be decontaminated in solution or suspension in the presence of a photoactive substance, a photosensitizer. Only the photodynamic method according to European Patent 0 491 757-B1 (H. Mohr and B. Lambrecht, Process for inactivating viruses in blood and blood products) is currently in widespread use. It is used for inactivating viruses in fresh plasma. The phenothiazine dye methylene blue is mainly used as the photoactive substance in the technical application. It is furthermore known that leukocytes in blood products can be killed by UV irradiation. In thrombocyte suspensions UV-B irradiation (wavelength range 290-320 mm) has proved suitable for this purpose.

Among others, the following important blood products for therapeutic applications are produced from blood donations: fresh plasma (FP), erythrocyte concentrates (EC) and thrombocyte concentrates (TC). As a rule, FP and EC are single-donor preparations whilst TC mostly consist of pooled thrombocytes from 4 to 6 single blood donations. Thrombocyte concentrates are the subject matter of the present invention.

In order to produce the afore-mentioned blood products, the blood donations are first centrifuged at high speed (at approximately 2000 to 5000 g, preferably approximately 4000 g, g=acceleration due to gravity); in this case the blood component are separated. The specifically heavier erythrocytes accumulate at the bottom. Above this is a narrow layer, the so-called “buffy coat” in which the white blood cells (granulocytes, monocytes and lymphocytes) and the thrombocytes are enriched. Above this again the plasma is located as a separate layer. The three components (EC, FP and the buffy coat) are then transferred into separate plastic bags. In order to produce TC, 4 to 6 buffy coats are pooled, suspended in plasma or a special storage medium and centrifuged at low speed. In this case, the white blood cells and the residual erythrocytes are pelleted whilst the thrombocytes are located in the supernatant, either in plasma or suspended in storage medium; in the latter case, the plasma concentration is generally between about 30 and 40%. This plasma content is currently regarded as necessary so that the TC can be stored.

Another method of obtaining TC from blood donations consists in centrifuging the blood donation at low speed (up to 1000 g, e.g. at about 300 g) so that the thrombocytes remain in the supernatant plasma. As a result of a further centrifugation step, these can be pelleted and then resuspended in a smaller volume of plasma or in storage medium with a certain plasma content.

Thrombocyte concentrates can also be obtained by mechanical thrombapheresis from donors, from whom only the thrombocytes and a small quantity of plasma in which the thrombocytes are suspended, are taken in this case.

In general, no importance is attached to white blood cells (leucocytes) in the blood products; they are even considered to be undesirable since they contain viruses or bacteria and can themselves trigger side effects in the recipients of blood products.

For example, leukocytes can bring about the formation of antibodies to foreign HLA antigens. As a result of this so-called allo-immunisation the recipients become refractory to transfusions of further TC from foreign donors.

Another problem is the graft versus host (GvH) reactions which are triggered by cytotoxic T-lymphocytes.

Attempts are made to eliminate said problems by removing the leukocytes by filtration. In the case of TC the depletion filters used for this purpose are not sufficiently effective however.

The irradiation of TC with UV-B light (wavelength range: approximately 290-320 nm) certainly inactivates leukocytes so that the risk of GvH reactions and allo-immunisation are reduced but these risks cannot be eliminated completely. This is only achieved by treatment with gamma radiation or x-rays. However, the technical and safety expenditure for this is so high that routine treatment of all preparations is out of the question.

Another problem with TC is potential contamination with bacteria which can enter into the products mainly as a result of the blood donation itself but also from its processing after the donation. In general, the extent of germination is initially low. In FP and EC which are stored deep frozen or at 4 to 8° C., the bacteria cannot multiply to a critical extent during storage. Thrombocyte concentrates on the other hand must be stored at about 20° C. so that the viability and functionality of the thrombocytes is retained. The TC storage time currently permitted is up to 5 days and in this time bacteria can multiply very strongly and can trigger serious septic reactions if infected preparations are transfused. According to studies, approximately 0.1% of all individual donors, or approximately 0.7% of all pool TC is contaminated with bacteria. These are mainly gram positive skin bacteria such as Staphylococcus (S.) epidermidis, S. aureus and Bacillus cereus; however, other bacteria have also been identified in TC, e.g. Streptococci and Klebsiella (the latter are gram negative).

According to estimates, in TC transfusion there is one fatal case of sepsis in one out of 320,000 to 700,000 cases. At the present time, the bacterial risk with blood products (FP, EC and especially TC) is considered to be higher than the viral risk. The object of the invention is thus to find a method with which leukocytes, especially T lymphocytes as well as bacteria in thrombocyte suspensions, especially TC, can be inactivated simultaneously without the function of thrombocytes and other blood components and their storage capability being impaired. In addition, as far as the effectiveness of the method is concerned, the plasma concentration in the thrombocyte suspensions should not play any role.

It has now surprisingly been found that the synthesis of photoactive substances can be induced in leukocytes and in bacteria, but clearly not in thrombocytes. The photoactive substances hereby accessible are called endogenous photosensitizers. The method according to the invention is defined by claim 1. Advantageous embodiments of the method are the subject matter of the dependent claims.

The thrombocyte suspensions used according to the invention are purified, concentrated blood products obtained from blood, if necessary also only indirectly, and are obtainable from blood or blood products as described previously for TC. The thrombocytes in the thrombocyte suspensions can be suspended, for example, in plasma or in a thrombocyte storage medium with arbitrary plasma content. Thrombocyte suspensions which have a thrombocyte concentration of more than 5×10⁸ thrombocytes per ml, especially preferably more than 10⁹/ml, are designated as TC.

If the preparations pre-treated with precursor compounds are irradiated using light of a suitable wavelength, this results in killing/deactivation of leukocytes and bacteria. The reason for this is seen in the fact that the photosensitizers are activated by the emitted light and now themselves activate dissolved oxygen molecules. Formed among other things are singlet oxygen, oxygen radicals, hydroxide anion radicals etc., which are all cytotoxic. The synthesis site of the endogenous photosensitizers in animal cells are the mitochondria and in bacteria, the cytosol. Surprisingly, the cytotoxic effect remains limited to the areas in which the photosensitizer was synthesized and has accumulated.

The precursor compounds are not themselves photoactive in the fashion described above but function as starting compounds which only require the further transformation, e.g. enzymatic transformation in order that photoactive substances are synthesised.

The photosensitizers whose endogenous synthesis can be induced preferably comprise porphyrin compounds such as protoporphyrin IX (PPIX) and coproporphyrin (CP).

The synthesis of PPIX takes place in the mitochondria of animal cells and in a number of bacteria using the following known scheme:

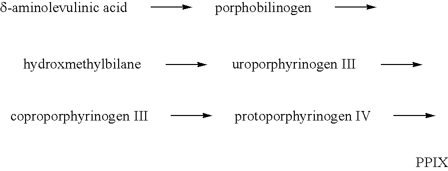

Thrombocytes have mitochondria like animal cells. It was thus to be expected that the synthesis of photoactive porphyrins will also be induced in them if they are incubated in the presence of delta-Ala. Said thrombocytes would thus be photosensitized like animal cells and consequently inactivated or damaged when illuminated. However, it was surprisingly established that functional in-vitro properties of TC (e.g., the hypotonic shock reaction and the aggregation induced by collagen for example) as well as other thrombocyte parameters had not changed after treatment with delta-Ala and subsequent exposure to light.

In most bacteria, no PPIX but CP and a number of other porphyrin compounds having a similar structure and having similar photochemical properties, are formed via a similar synthesis path. Both in animal cells and also in bacteria the synthesis starts with 6-aminolevulinic acid (delta-Ala) which is itself synthesised intracellularly wherein glycine and succinyl coenzyme A are the preliminary stages. In the case of delta-Ala or its derivatives, the enzymatic transformation takes place after passing membrane barriers intracellularly.

Delta-Ala is thus an endogenous substance; its plasma concentration in healthy individuals is between 0.024 and 0.27 μM/L according to published investigations.

It has been known for a long time that the synthesis of PPIX or CP both in activated cells and in bacteria can be increased substantially if delta-Ala is supplied to them as substrate. A plurality of concepts for the photodynamic therapy of tumours and inflammatory diseases, of the skin for example, are based thereon. There are also concepts for the photodynamic therapy of bacterial infections (see, for example, “Photodynamic destruction of Haemophilus parainfluenzae by endogenously produced porphyrins” by van der F. W. Meulen, K. Ibrahim, H. J. Sterenborg, L. V. Alphen, A. Maikoe, J. Dankert in Photochem. Photobiol. N 1997 October; 40(3), 204-8).

Tumour cells or cells in inflamed tissue are generally more strongly activated than non-degenerate body cells and accordingly synthesise considerably more PPIX when delta-Ala is supplied to them

In general, however, peripheral blood lymphocytes (PBL) are in the quiescent state and it was thus to be expected that the synthesis of PPIX to an extent sufficient for photodynamic killing cannot be induced in them by delta-Ala. This is deduced among others from the following publications: D. Grebenova et al., (1998) “Selective destruction of leukaemic cells by photo-activation of 5-aminolevulinic acid-induced protoporphyrin-IX”, J. Photochem. Photobiol. B.: Biol. 47, 74-78 and E. A. Hryorenko et al. (1998), “Characterization of endogenous protoporphyrin IX induced by delta-aminolevulinic acid in resting and activated peripheral blood lymphocytes by fourcolor flow cytometry,” Photochem. Photobiol., 67, 565-572.

In the present case which involves the treatment of thrombocyte suspensions approximately at room temperature and not at the optimal temperature of 37° C. for the proliferation of cells, this appeared particularly unlikely. According to publications, in the presence of plasma proteins and other plasma components, the intracellularly synthesised porphyrins additionally accumulate not in the cells but are locked out where they are ineffective because their concentration is too low and because plasma components have an inhibiting effect (see, for example, J. Hanania, Z. Mailk “The effect of EDTA and serum on endogenous porphyrin accumulation and photodynamic sensitization of human K562 leukemic cells” Cancer Lett. 65(1992), 127-131).

It thus appeared improbable that bacteria are inactivatable in thrombocyte concentrates because, as has already been mentioned, a minimum concentration of about 30% plasma is presently required so that they retain their functionality during storage. It was thus very surprising that under the conditions described, both peripheral blood lymphocytes and also bacteria are inactivatable or can be killed at all following pre-treatment with delta-Ala and subsequent exposure to light.

The treatment of thrombocyte suspensions according to the invention is preferably carried out as follows:

Blood donations are usually stored in the form of packaging units having contents of 450 to 500 ml in special plastic bags. The thrombocyte suspensions concerned are also located in quantifies of about 100 to 1000 ml, preferably about 200 to 600 ml, in a transparent bag made of plastic film, e.g. made of PVC or polyolefins such as are usually used for the production and storage of said blood products.

Delta-Ala is added to the thrombocyte suspension in the required concentration; it is then incubated for a pre-determined time at a temperature which makes it possible for delta-Ala to penetrate into leukocytes and bacteria, and also for a sufficient porphyrin synthesis.

In the experiments carried out, incubation was generally carried out at room temperature but higher or lower temperatures are also possible. It is important that delta-Ala can penetrate into the target cells, that PPIX and other porphyrins are synthesised and that FP or thrombocytes or erythrocytes are not damaged at the relevant temperatures.

Instead of delta-Ala it is also possible to use derivatives of delta-Ala e.g. its esters or amides which can probably penetrate into the cellular membranes and be taken up by cells more easily than delta-Ala itself because of their more lipophilic nature, especially those whose alcohol or amide group has 1 to 4 carbon atoms. When using more lipophilic derivatives of delta-Ala however, it should be borne in mind these do not concentrate very easily in thrombocytes for example. Suitable delta-Ala derivatives and their synthesis are described in the citation J. Kloek; G. M. J. Beijersbergen van Henegouwen; “Prodrugs of 5-aminolevlinic acid for photodynamic therapy”; Photochemistry and Photobiology 64(6), 1964, 994-1000 which is hereby also made the contents of this application. As a precaution, it may be mentioned that whenever reference is made to delta-Ala in the present application, a delta-Ala derivative can also be used or is meant instead of delta-Ala. The concentration of delta-Ala used depends primarily on the plasma concentration of the blood product to be treated. For example, in the case of TC which are frequently suspended in almost 100% plasma as suspending medium, the effective concentration of delta-Ala is between about 0.5 and 5 mM (M=mol/l). If the TC are suspended in a storage medium with only about 30% plasma, approximately 100 to 1000 μM delta-Ala is sufficient.

The afore-mentioned pre-incubation to accumulate PPIX and other porphyrins can last between a few minutes and about. 20 hours depending on the temperature and desired effect. If only bacteria having a high dividing activity are to be inactivated for example, approximately 15 min is sufficient at room temperature.

If white blood cells are also be included, which are generally located in the quiescent phase of the cell cycle, absorb little material and have a low metabolism, the pre-incubation duration must be extended to several hours, e.g. over 16 hours. This is no problem with TC since these are stored at room temperature in any case.

Following the pre-incubation the thrombocyte suspensions are exposed to light. As far as the type of light sources used is concerned, there are several possibilities since PPIX, CP and the other porphyrin compounds formed intracellularly have a plurality of light absorption maxima in the visible and in the ultraviolet part of the spectrum. One absorption maximum of PPIX lies at 635 nm, that is in the red, whilst other maxima lie in the visible range between approximately 400 and 580 nm; in the long-wavelength UV range (UV-A) there is the largest maximum between approximately 320 and 400 nm. The situation is similar with CP which is synthesized by bacteria, as mentioned. Radiation having wavelengths in the range from 320 to 580 nm, especially preferably from 320 to 500 nm, is preferably used. Radiation which exclusively has light at wavelengths above 580 nm has surprisingly proved less effective for thrombocyte suspensions.

However, the light absorption maximum of CP in the red part of the spectrum does not lie at 635 but at 617 nm. Thus, it was then also established that bacteria exposed to light at this wavelength are inactivated more effectively than at 635 nm (F. W. van der Meulen et al. (1997): “Photodynamic destruction of Haemophilus parainfluenzae by endogenously produced porphyrins”, J. photochem. Photobiol. B: Biol. 40, 204-208). In our own studies on the inactivation of bacteria in TC however, neither of the two wavelengths was found to be sufficiently effective when used substantially exclusively. However, it was surprisingly found that bacteria and also leukocytes in TC after pre-treatment with delta-Ala can be effectively inactivated if they are irradiated with white light, such as emitted by xenon lamps for example. This is demonstrated in the following experimental examples.

Light sources which can be used, can emit red, green, blue, white, UV-A or UV-A and white light, as is the case with xenon lamps for example. If the UV-A fraction of the absorption spectrum is to be used, care should be taken to ensure that the film material from which the plastic containers used are made, is UV-transparent.

The exposure time depends on the product which is being treated, i.e., on its permeability to light and on that of the film material of the plastic bag in which it is located, on the light source used (the higher its intensity, the shorter the exposure time required), on the delta-Ala concentration and on the pre-incubation duration and temperature in the presence of delta-Ala before the exposure, i.e., on the quantity of PPIX and other porphyrins which have been formed in the target cells or bacteria and have become enriched.

For these reasons, it is not easily possible to make a general statement on the required exposure time regardless of the material used and exposure should be continued until the target cells and micro-organisms have been killed to the required extent.

Materials and Methods

In the experiments described, TC was used which had been obtained from the buffy coats of donor blood and which was suspended in plasma or in a conventional storage medium (T-sol) with approximately 40% plasma. A system fitted with xenon lamps and in which the emitted UV fraction was masked out by a window glass filter was used for the exposure. Staphylococcus (S.) epidermidis was generally used as the test bacterium. Bacteria titres were determined using a colony forming assay. These are given in “colony forming units per ml” (CFU/ml).

Mononuclear cells (MNC) were isolated by means of density gradient centrifugation over Ficoll®/Hypaque® from donor blood. In the investigations on the inactivation of leukocytes these cells were added to the TC in a concentration of 7×10⁵/ml. After the treatment aliquots of the cell suspensions were centrifuged at low speed (1500 rpm or 600 g for 4 min).

The pelleted cells were washed three times with cell culture medium (RPMI 1640 with 10% foetal calf serum and antibiotic) and were then resuspended in a cell concentration of 7×10⁵/ml again in the same medium. The vitality of the cells was checked by means of a proliferation assay. In this case, the cells were stimulated with Concanavalin A (Con A, 2 μg/ml) and were cultivated in 200 μl aliquots for 3 to 4 days at 37° C. in a CO₂-gassed breeding cabinet. Bromodeoxyuridine (BRDU) was then added to the cell suspensions. These were then cultivated for a further 4 hours and thereafter the incorporation rate of BRDU was determined spectrophotometrically at a wavelength of 450 nm. The extinction values (OD₄₅₀) at this wavelength are proportional to the incorporation of BRDU into the cellular DNA and thus to the viability of the cells.

### EXPERIMENTAL EXAMPLES

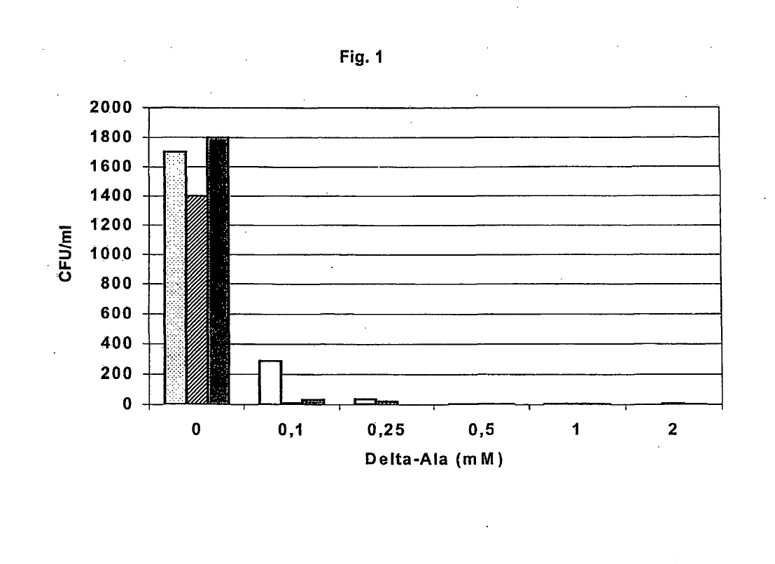

In the experiments on the inactivation of bacteria TC aliquots of respectively 60 ml located in PVC storage bags having a nominal volume of 500 ml were treated. After adding delta-Ala or the methyl ester of delta-Ala (see Table 1) the samples were pre-incubated for one hour at room temperature in the thrombocyte rotator and then exposed to light. In the experiments on the inactivation of leukocytes the samples were pre-incubated overnight (i.e., for approximately 16-20 h) or for 4 hours. The light energy deposited in the samples is given in kilojoules per m² (kJ/m²). 3000 kJ/m² corresponds to an exposure time of about one hour. Each experiment was carried out at least twice. The results of the experiments are given in Tables 1 and 2 and plotted in FIGS. 1 to 5. The results of repetitions of the experiments are indicated by different colours of the bars.

Photoinactivation of Bacteria

Dependence on the Delta-Ala Concentration

FIG. 1 shows the dependence of the photoinactivation of S. epidermidis in TC on the delta-Ala concentration. The light energy input was 3000 kJ/m².

As can be seen from the result, S. epidermidis is extensively inactivated when TC is pre-incubated with delta-Ala in the concentration range between approximately 0.5 and 1 mM and then intensively exposed to light. In plasma-reduced TC delta-Ala concentrations of 0.1 mM or less were already sufficient under otherwise the same experimental conditions. The results of three experiments are plotted.

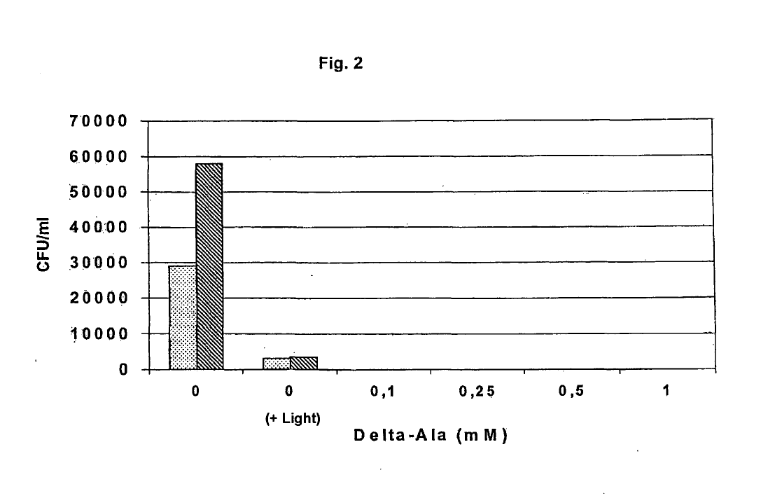

FIG. 2 shows the dependence of the photoinactivation of S. epidermidis in plasma-reduced TC on the delta-Ala concentration. The light energy input was again 3000 kJ/m². The results of two experiments are plotted. The exposure to light alone resulted in a significant reduction in the bacteria titre.

Kinetics of the Photoinactivation of S. epidermidis

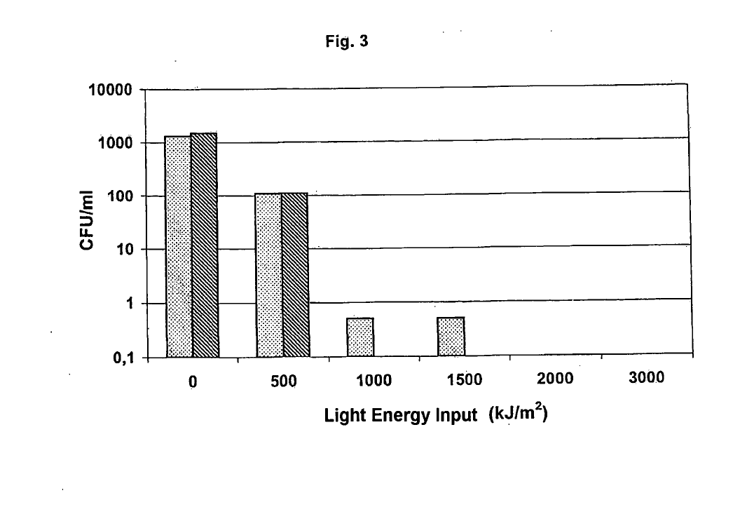

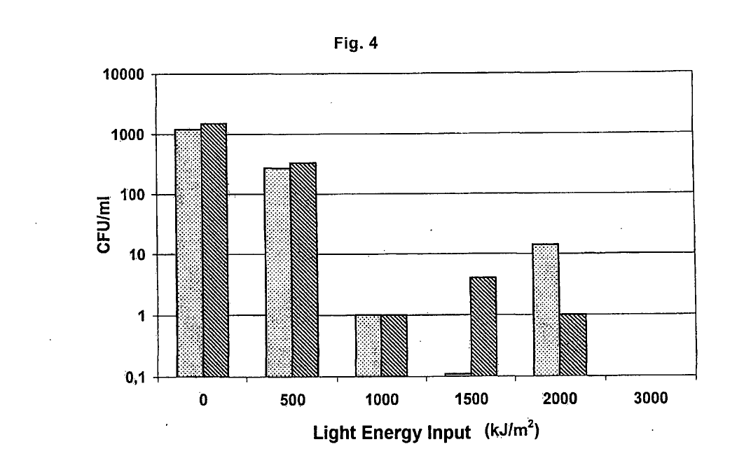

In the experiments whose results are plotted in FIGS. 1 and 2, the TC was exposed to 3000 kJ/m² which is certainly more than is actually required. In order to determine the quantity of light sufficient to largely inactivate S. epidermidis, i.e., by at least 2 log₁₀ grades, the following experiments were carried out: 1000 to 1500 CFU/ml S. epidermidis was added to TC, either in storage medium having a plasma content of approximately 40% or in plasma; delta-Ala was then added in a concentration of 0.25 mM (plasma-reduced TC) or 1 mM (TC with approximately 100% plasma). After pre-incubating for one hour at room temperature, the samples were exposed to different amounts of light. The results are plotted in FIGS. 3 and 4.

The kinetics of the photoinactivation of S. epidermidis in plasma-reduced TC after pre-incubation for one hour in the presence of 0.25 mM delta-Ala is shown in FIG. 3.

The kinetics of the photoinactivation of S. epidermidis in TC after pre-incubation for one hour in the presence of 1 mM delta-Ala is shown in FIG. 4.

It can be concluded from FIGS. 3 and 4 that for the plasma-reduced TC approximately 1500 to 2000 kJ/m² was sufficient for extensive inactivation of the bacteria whereas for thrombocytes which were suspended in 100% plasma, approximately 3000 kJ/m² of light should be irradiated.

Use of the Methyl Ester of Delta-Ala Instead of Delta-Ala

It can be seen from Table 1 that the methyl ester of delta-Ala has the same efficacy as far as the inactivation of S. epidermidis is concerned. This confirms that derivatives of delta-Ala are likewise converted into photoactive substances and that this can also be used for the intended purpose i.e., for decontamination of blood components.

TABLE 1
Photoinactivation of S. epidermidis in TC in 100% plasma.
Comparison of delta-Ala with delta-Ala methyl ester (methyl-Ala).
Both compounds were used in a concentration of 2 mM. Light
energy input: 3000 kJ/m². The control sample was not
exposed to light.

Bacteria titre after light
(CFU/ml)

| Compound added | Exp. 1 | Exp. 2 |
|---|---|---|
| Control | 250 | 210 |
| Delta-Ala | 5 | 3.5 |
| Methyl-Ala | 1.5 | 4 |

Photoinactivation of Further Bacteria

It can be deduced from Table 2 that in addition to S. epidermidis, other gram-positive and gram-negative bacteria in TC cam also be inactivated if they are pre-treated with delta-Ala and then exposed to light. E. cloacae and S. aureus are clearly somewhat more resistant than the other test bacteria; however, even for these the infectious titre was reduced by approximately 95 or 97% under the selected experimental conditions. It can be deduced from the results of the previous studies that all bacteria can be completely inactivated if the exposure time is lengthened or the concentration of delta-Ala is increased.

TABLE 2
Photoinactivation of various bacteria in TC after pre-treatment with
1 mM delta-Ala (averages of respectively two experiments), light
energy input: 1500 kJ/m².

| | | Bacteria titre (CFU/ml) | |
|---|---|---|---|
| Bacterium | Gram staining | before exposure/after exposure | |
| Staphylococcus | + | 2440 | 2 |
| epidermidis | | | |
| Pseudomonas | − | 700 | 1 |
| aeroginosa | | | |
| Staphylococcus | + | 2000 | 51 |
| aureus | | | |
| Yersinia | − | 4650 | 1 |
| enterocolitica | | | |
| Escherichia coli | − | 11300 | 3 |
| Serratia marescens | − | 2500 | 1 |
| Enterobacter cloacae | − | 24500 | 1280 |

Photoinactivation of White Blood Cells
Dependence on the Delta-Ala Concentration

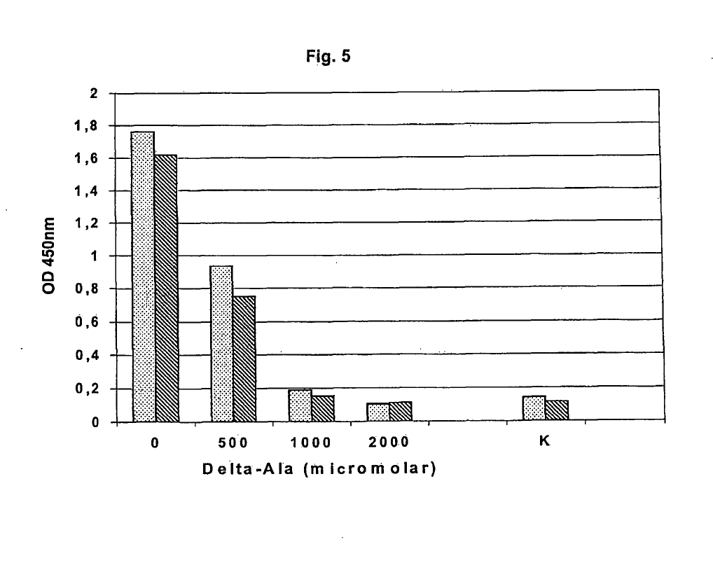

FIG. 5 shows the photoinactivation of T lymphocytes in TC as a function of the concentration of delta-Ala (concentration range: 500-2000 μM, pre-incubation time: approx. 16 h; light energy input: 2000 kJ/m², K=unstimulated control sample). The required concentration of delta-Ala is clearly higher than 1 mM. Unlike bacteria which only need to be pre-incubated for a short time (in the preceding studies the pre-incubation time was always one hour; but 15-30 minutes was sufficient), it is generally necessary to extend the pre-incubation time to several hours so that the cells can be largely inactivated by a light energy input of 1000-2000 kJ/cm².

1. A method for inactivating bacteria and/or leukocytes in thrombocyte suspensions obtainable from blood or blood products by purification and concentration, by photodynamic treatment comprising the following steps:
(a) Preparing a thrombocyte suspension, wherein said thrombocyte suspension is a thrombocyte concentrate which contain at least 5×10⁸ thrombocytes per ml,
(b) Adding at least one precursor substance for the synthesis of at least one photoactive substance, wherein said precursor substance is at least δ-aminolevulinic acid or a derivative of δ-aminolevulinicacid,
(c) Inducing intracellular synthesis of one or a plurality of photoactive substances and
(d) Exposing said thrombocyte suspension to irradiation in at least one absorption wavelength range of the photoactive substance(s).

2. The method according to claim 1, wherein said precursor substance is an ester or amide of δ-aminolevulinic acid.

3. The method according to claim 1, characterized in that said precursor substance is added in a quantity such that a concentration of 0.01 to 10 mmol per liter, is formed.

4. The method according to claim 1, characterized in that said precursor substance is added at least 15 minutes prior to the irradiation.

5. The method according to claim 1, characterized in that said irradiation is carried out using light in the range of wavelengths of at least one absorption maximum, of said photoactive substances whose synthesis was induced.

6. The method according to claim 1, characterized in that said irradiation is carried out using light, in the range of wavelengths from 320 nm to 580 nm.

7. The method according to claim 1, characterized in that said thrombocyte suspension together with said precursor substance is incubated at 15 to 28° C., to form the photoactive substance(s).

8. The method according to claim 1, characterized in that said irradiation is carried out using light energy of 100 to 10,000 kJ/cm².

9. The method according to claim 1, characterized in that said thrombocyte suspensions contain plasma.

10. The method according to claim 1, characterized in that said treatment of the thrombocyte suspensions takes place in blood bags.

11. The method according to claim 9, characterized in that said treatment of said thrombocyte suspension takes place in a continuous flow apparatus.

12. The method according to claim 1, characterized in that said leukocytes are T lymphocytes.

13. A method for inactivating leukocytes in thrombocyte suspensions obtainable from blood or blood products by purification and concentration, by photodynamic treatment comprising the following steps:
(a) Preparing a thrombocyte suspension, wherein said thrombocyte suspension is a thrombocyte concentrate which contains at least 5×10⁸ thrombocytes per ml,
(b) Adding at least one precursor substance for the synthesis of at least one photoactive substance, wherein said precursor substance comprises at least δ-aminolevulinic acid or a derivative of δ-aminolevulinic acid,
(c) Inducing intracellular synthesis of one or a plurality of photoactive substances and
(d) Exposing said thrombocyte suspension to irradiation in at least one absorption wavelength range of said photoactive substance(s), wherein the treatment comprises at least light in the wavelength range from 320 nm to 580 nm.

14. A method for inactivating bacteria in thrombocyte suspensions obtainable from blood or blood products by purification and concentration, by photodynamic treatment comprising the following steps:
(a) Preparing a thrombocyte suspension, wherein said thrombocyte suspension is a thrombocyte concentrate which contains at least 5×10⁸ thrombocytes per ml,
(b) Adding at least one precursor substance for the synthesis of at least one photoactive substance, wherein said precursor substance comprises at least δ-aminolevulinic acid or a derivative of δ-aminolevulinic acid,
(c) Inducing intracellular synthesis of one or a plurality of photoactive substances and
(d) Exposing said thrombocyte suspension to irradiation in at least one absorption wavelength range of said photoactive substance(s), wherein the treatment comprises at least light in the wavelength range from 320 nm to 580 nm.

15. The method according to claim 3, characterized in that said precursor substance is added in a quantity such that a concentration between 0.1 and 5 mmol per liter is formed.

16. The method according to claim 4, characterized in that said precursor substance is added at least 1 hour prior to the irradiation.

17. The method according to claim 7, characterized in that said thrombocyte suspension together with said precursor substance is incubated at 18 to 25° C.

18. The method according to claim 8, characterized in that said irradiation is carried out using light energy of 100 to 3000 kJ/cm².

19. The method according to claim 18, characterized in that said irradiation is carried out using light energy of 500 to 2000 kJ/m².

20. The method according to claim 5, characterized in that said irradiation is carried out using light in the range of wavelengths of a plurality of absorption maxima of said photoactive substances whose synthesis was induced.

21. The method according to claim 5, characterized in that said irradiation is carried out comprising light in the range of wavelengths from 320 nm to 580 nm.

22. The method according to claim 6, characterized in that said irradiation further comprises monochromatic light.

23. The method according to claim 9, wherein said thrombocyte suspensions further comprises a suitable storage medium.

24. The method according to claim 9, wherein said plasma is present in an amount higher than 20%.

25. The method according to claim 13 wherein exposing of said thrombocyte suspension to irradiation further comprises exposure to monochromatic light in the wavelength range from 320 nm to 580 nm.

26. The method according to claim 14 wherein exposing said thrombocyte suspension to irradiation further comprises exposure to monochromatic light in the wavelength range from 320 nm to 580 nm.
